(19) United States
(12) Patent Application Publication
(10) Pub. No.: US 2014/0172317 A1
(43) Pub. Date: Jun. 19, 2014

(54) METHOD AND APPARATUS FOR PERFORMING DRUG SCREENING

(71) Applicant: Samsung Electronics Co., Ltd., Suwon-si (KR)

(72) Inventors: Jung-joon LEE, Seoul (KR); Sang-ok SONG, Seongnam-si (KR); Kyoung-hu LEE, Hwaseong-si (KR)

(73) Assignee: Samsung Electronics Co., Ltd., Suwon-si (KR)

(21) Appl. No.: 14/104,660

(22) Filed: Dec. 12, 2013

(30) Foreign Application Priority Data
Dec. 13, 2012 (KR) 10-2012-0145706

(51) Int. Cl. G01N 33/50; G06F 19/22

(52) U.S. Cl. CPC G01N 33/5008; G06F 19/22
USPC 702/19

(57) ABSTRACT

Provided are a method and apparatus for performing drug screening. The method includes: obtaining statistical data regarding prognostic indices that indicate a recurrence likelihood of a disease and expression levels of phenotype features from biological samples; obtaining drug data regarding expression levels of the determined phenotype features that are changed by administering different types of drugs to the biological samples; and screening efficacy of the administered drugs by using the obtained statistical data and the obtained drug data.

### CROSS-REFERENCE TO RELATED APPLICATIONS

This application claims the benefit of Korean Patent Application No. 10-2012-0145706, filed on Dec. 13, 2012, in the Korean Intellectual Property Office, the entire disclosure of which is incorporated herein by reference.

### BACKGROUND

1. Field

The present disclosure relates to methods and apparatuses for performing drug screening, whereby a drug having efficacy on a particular disease is screened.

2. Description of the Related Art

Techniques for analyzing genes of biological samples, such as a patient's cells, have been continuously developed since deoxyribonucleic acid (DNA), a nucleic acid, has been discovered. In general, it is well known that similar gene expression patterns in various experimental conditions are shown in genes having biologically similar functions or a high biological correlation. Gene expression profiles can be obtained by measuring gene expression levels of genes of biological samples according to a change of several experimental conditions using the above well-known fact.

In particular, these gene expression profiles are mainly used in developing new drugs or checking gene expression levels or gene expression patterns of cells when a patient's disease is cured. Techniques for inspecting features relating to diseases by using an imaging technique, such as high throughput screening, have recently emerged. However, techniques for precisely discovering features that clinically relate to diseases among many features relating to diseases are under study.

### SUMMARY

Provided are methods and apparatuses for performing drug screening.

Additional aspects will be set forth in part in the description which follows and, in part, will be apparent from the description, or may be learned by practice of the presented embodiments.

According to an aspect of the present disclosure, a method of performing drug screening includes: receiving or obtaining statistical data regarding prognostic indices that indicate a recurrence likelihood of a disease and expression levels of phenotype features from biological samples; determining phenotype features in which a correlation exists by analyzing the correlation between the obtained prognostic indices and the obtained expression levels; receiving or obtaining drug data regarding expression levels of the determined phenotype features that are changed by administering different types of drugs to the biological samples; and screening efficacy of the administered drugs using the analyzed correlation and the obtained drug data. The method steps are typically implemented in one or more processors.

According to another aspect of the present disclosure, a method of performing drug screening includes: analyzing a correlation between prognostic indices that indicate a recurrence likelihood of a disease and expression levels of phenotype features on biological samples; receiving or obtaining data regarding expression levels of the phenotype features that are changed by administering different types of drugs to the biological samples; and screening the administered drugs by predicting a change of the prognostic indices that correspond to a change of the phenotype features analyzed that the correlation exits in the obtained data, by using the analyzed correlation. The method steps are typically implemented in one or more processors.

According to another aspect of the present disclosure, there is provided a non-transitory computer-readable recording medium having recorded thereon a program for executing the method.

According to another aspect of the present disclosure, an apparatus for performing drug screening includes: a data obtaining unit that receives or obtains statistical data regarding prognostic indices that indicate a recurrence likelihood of a disease and expression levels of phenotype features from biological samples; a phenotype feature analyzing unit that determines phenotype features in which a correlation exists by analyzing the correlation between the obtained prognostic indices and the obtained expression levels; and a drug screening unit that screens efficacy of administered drugs by using the analyzed correlation and drug data regarding expression levels of the determined phenotype features that are changed by administering different types of drugs to the biological samples, wherein the drug data is obtained by the data obtaining unit.

According to another aspect of the present disclosure, an apparatus for performing drug screening includes: a phenotype feature analyzing unit that analyes a correlation between prognostic indices that indicate a recurrence likelihood of a disease and expression levels of phenotype features on biological samples; a data obtaining unit that receives or obtains data regarding expression levels of the phenotype features that are changed by administering different types of drugs to the biological samples; and a drug screening unit that screens the administered drugs by predicting a change of the prognostic indices that correspond to a change of the phenotype features analyzed that the correlation exits in the obtained data, using the analyzed correlation.

### BRIEF DESCRIPTION OF THE DRAWINGS

These and/or other aspects will become apparent and more readily appreciated from the following description of the embodiments, taken in conjunction with the accompanying drawings in which:

FIG. 1 illustrates a drug screening system according to an embodiment of the present disclosure;

FIG. 2 is a detailed block diagram of a structure of an apparatus for performing drug screening, according to an embodiment of the present disclosure;

FIG. 3 is a table showing cell lines that may be used as biological samples, according to an embodiment of the present disclosure;

FIG. 4 is a table showing phenotype features analyzed by an expression level analyzing unit, according to an embodiment of the present disclosure;

FIG. 5 is a graph for explaining an operation of analyzing a correlation between prognostic indices and phenotype features by using a phenotype feature analyzing unit, according to an embodiment of the present disclosure;

FIG. 6 is a graph showing drug data regarding expression levels of phenotype features that are changed and determined by administering different types of drugs to biological samples, according to an embodiment of the present disclosure;

FIG. 7 is a flowchart illustrating a method of performing drug screening, according to an embodiment of the present disclosure; and

FIG. 8 is a flowchart illustrating a method of performing drug screening, according to another embodiment of the present disclosure.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. Expressions such as “at least one of,” when preceding a list of elements, modify the entire list of elements and do not modify the individual elements of the list.

FIG. 1 illustrates a drug screening system 1 according to an embodiment of the present disclosure. Referring to FIG. 1, the drug screening system 1 according to the current embodiment of the present disclosure includes an apparatus 10 for performing drug screening, biological samples 20 before drug administration is performed, and biological samples 30 after drug administration is performed.

Although not shown, one of ordinary skill in the art understands that the drug screening system 1 may further include a gene analyzing device, such as a polymerase chain reaction (PCR) device for detecting phenotypes or gene expression levels from the biological samples 20 or 30, a microarray, a high content cell imaging device, a high content screening device, and a high throughput screening device.

That is, the drug screening system 1 illustrated in FIG. 1 illustrates elements relating to the present embodiment to not make the features of the present embodiment vague. However, the drug screening system 1 may further include other general-use elements than elements illustrated in FIG. 1.

In FIG. 1, the biological samples 20 and 30 are separately illustrated so as to distinguish between the time before drug administration is performed and the time after drug administration is performed but they are the same. The number of biological samples 20 illustrated is three for convenience of explanation of the present embodiment and may be changed according to the environment of the drug screening system 1.

A nucleic acid, such as deoxyribonucleic acid (DNA) of an object, corresponds to a gene substance including gene information about the object, i.e., a gene. A base sequence of the nucleic acid includes information about cells or tissue that constitutes the object. Thus, many studies for information about a thorough nucleic acid sequence of an individual have been performed in many fields, such as in understanding the phenomenon of life, in developing new drugs, in diagnosing and preventing a disease, and in studying genes of humans.

Compounds, such as drugs used as medicine for curing various diseases (for example, cancer, tumors, and the like) may affect part or the whole of a gene expression level. Thus, if the effect on the expression level of a gene can be precisely analyzed, the efficacy of the medicine, such as drugs, may be precisely monitored or predicted.

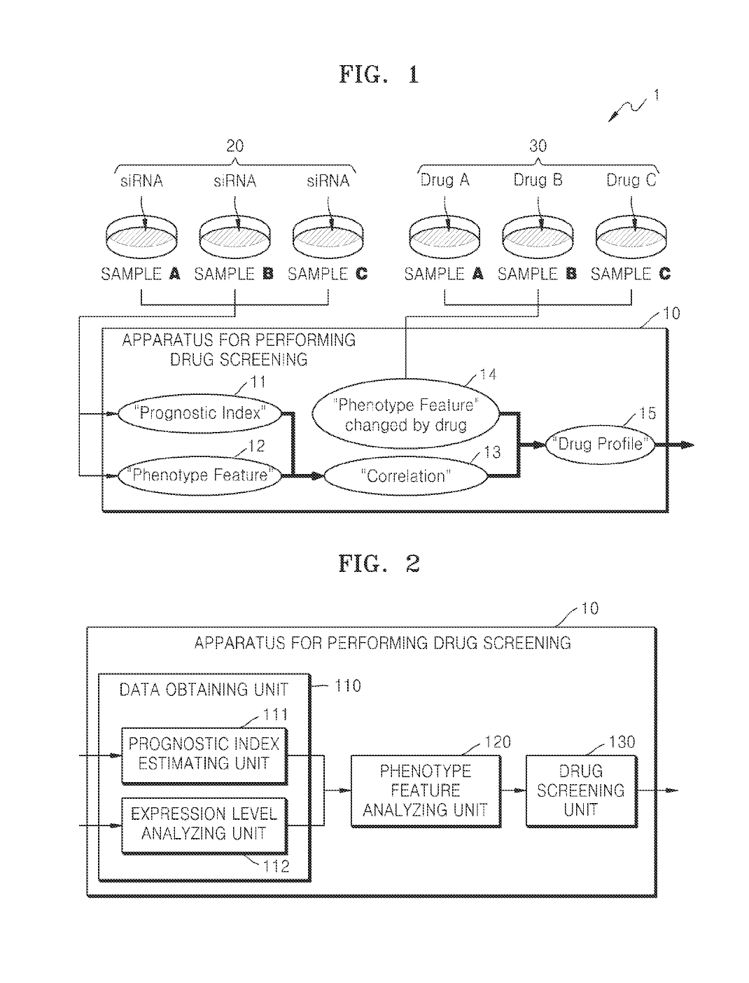

The apparatus 10 for performing drug screening of the drug screening system 1 of FIG. 1 is an apparatus for screening a drug having efficacy among drugs A, B, and C that are administered to the biological samples 20. Here, the biological samples 20 and 30 are samples including an abnormal tissue, such as a cancer or tumor extracted from a patient, that are cell samples, tissue samples, or serum samples, for example. Hereinafter, the operation of the drug screening system 1 will be described in detail together with reference to FIG. 2 which illustrates the apparatus 10 for performing drug screening.

FIG. 2 is a detailed block diagram of a structure of the apparatus 10 for performing drug screening, according to an embodiment of the present disclosure. Referring to FIG. 2, the apparatus 10 for performing drug screening includes a data obtaining unit 110, a phenotype feature analyzing unit 120, and a drug screening unit 130. The data obtaining unit 110 includes a prognostic index estimating unit 111 and an expression level analyzing unit 112.

The apparatus 10 for performing drug screening may be implemented with one or more general-use processors. That is, the apparatus 10 for performing drug screening may be implemented with an array of logic gates or a combination of one or more general-use microprocessors and memory in which a program that may be executed by the microprocessor(s) is stored. Also, the apparatus 10 for performing drug screening may be implemented in the form of a module of an application program. Furthermore, one of ordinary skill in the art understands that the apparatus 10 for performing drug screening may be implemented with hardware having other forms that may perform operations as described herein.

The data obtaining unit 110 obtains statistical data regarding prognostic indices 11 and expression levels of phenotype features 12 from the biological samples 20.

The prognostic index estimating unit 111 estimates prognostic indices 11 that indicate the recurrence likelihood of a disease, using gene expression data obtained from the biological samples 20.

In certain aspects, the gene expression data is obtained from each of the biological samples 20 using reverse transcriptase polymerase chain reaction (RT-PCR) or a microarray, or any of several other well-known methods of obtaining gene expression data. A method of obtaining gene expression data is obvious to one of ordinary skill in the art, and thus detailed descriptions thereof will be omitted.

The prognostic indices 11 that are estimated by the prognostic index estimating unit 111 may include a recurrence score, a tumor grade, and a C-path score.

In certain aspects, the recurrence score is a value between 0 and 100 that is obtained from the result of performing a diagnostic test, such as an Oncotype DX® test that diagnoses breast cancer by analyzing 21 different genes from tissue of the breast cancer. The recurrence score is an index that indicates the recurrence likelihood of breast cancer and the effective period of chemotherapy. As the recurrence score increases, the recurrence likelihood of breast cancer increases.

The tumor grade and the C-path score are also indices that predict a recurrence rate of cancer using a biopsy of the tissue of the breast cancer.

Although various methods of predicting the recurrence likelihood of a disease, such as a cancer or tumor, are well known in the art, the recurrence score is generally known as the prognostic indices 11 having a relatively precise result. In the present embodiment, the recurrence score has been explained as an example, and aspects of the present disclosure are not limited thereto. That is, one of ordinary skill in the art understands that any prognostic indices that indicate the recurrence likelihood of the disease may be used.

Samples A, B, and C that are the biological samples 20 before drug administration is performed may be samples that are transfected by small interfering RNA (siRNA) and thus have different gene expression levels.

In particular, when the recurrence score is used, the biological samples 20 may correspond to cell lines that are classified into a high risk group according to the recurrence score. However, the cell lines of the high risk group are not necessarily used as the biological samples 20.

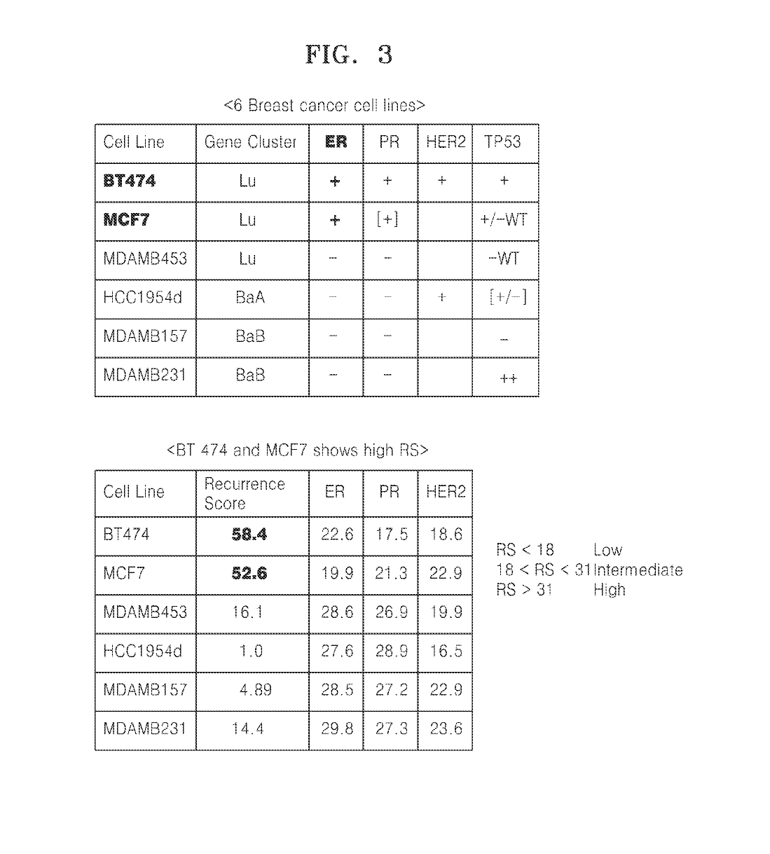

FIG. 3 is a table showing cell lines that may be used as the biological samples 20, according to an embodiment of the present disclosure.

Referring to FIG. 3, data regarding 6 cell lines of breast cancer of a human are shown. As a result of obtaining gene expression levels of 21 genes of the breast cancer tissue regarding a recurrence score, cell lines, such as BT474 and MCF7, belong to a high risk group and are ER positive. Thus, it may be determined that BT474 and MCF7 may be used as appropriate biological samples 20.

The biological samples 20, such as samples A, B, and C, may correspond to samples of different cell lines. That is, the biological samples 20 may correspond to different cell lines that are extracted from breast cancer cells, as described in FIG. 3. However, a reason why different cell lines are used is that the biological samples 20 have different gene expression levels. Thus, a similar result to that where the above-described biological samples 20 are transfected by siRNA may be obtained.

Referring back to FIG. 2, the prognostic index estimating unit 111 estimates recurrence scores of the biological samples 20. As described above, since the biological samples 20 are transfected by siRNA, they have different gene expression levels. Thus, the biological samples 20 may have recurrence scores that are not the same.

The expression level analyzing unit 112 analyzes the phenotype features 12 of the biological samples 20 using image data in which phenotypes expressed in the biological samples 20 are indicated.

In more detail, the expression level analyzing unit 112 obtains the image data regarding the phenotypes of the biological samples 20 from an imaging device, such as a high content cell imaging device, a high content screening device, or a high throughput screening device, and then analyzes the phenotype features 12 from the obtained image data. Here, before the image data is obtained, an operation of labeling the biological samples 20 with fluorescent dyes may be performed. The operation of obtaining the image data from the imaging device, such as the high content cell imaging device, the high content screening device, or the high throughput screening device, is well known to one of ordinary skill in the art and thus, detailed descriptions thereof will be omitted.

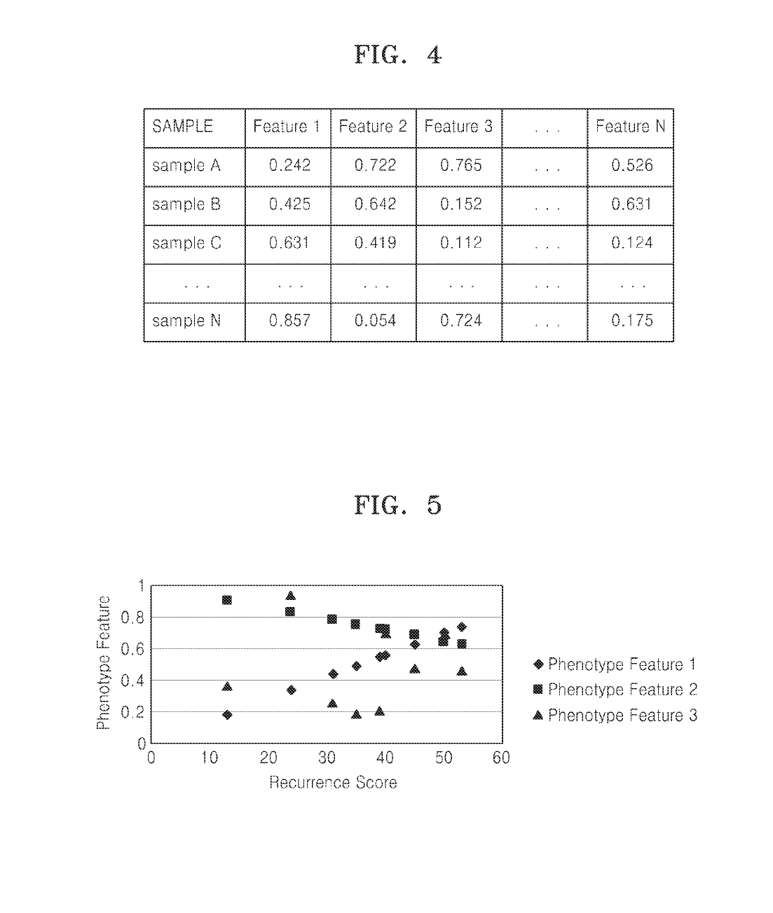

FIG. 4 is a table showing phenotype features 12 analyzed by the expression level analyzing unit 112, according to an embodiment of the present disclosure.

Referring to FIG. 4, at least one phenotype is expressed in each of the biological samples 20, and relative values therefrom are analyzed as expression levels of the phenotype features 12. In more detail, phenotypes of the biological samples 20 may be expressed in various types of image data, such as the intensity of brightness according to the result of hybridization of the fluorescent dyes, and the shape of a hybridized region. The expression level analyzing unit 112 analyzes expression levels of the phenotype features 12 by calculating a relative contribution of each of the phenotype features 12.

As a result, the expression level analyzing unit 112 analyzes the expression levels of the phenotype features 12 from each of the biological samples 20 so as to obtain statistical data.

Referring back to FIG. 2, the phenotype feature analyzing unit 120 analyzes a correlation 13 between the prognostic indices 11 and the expression levels of the phenotype features 12 on each of the biological samples 20. Then, the phenotype feature analyzing unit 120 determines phenotype features 14 in which the correlation 13 exists, among all phenotype features 12 that are analyzed by the expression level analyzing unit 112, based on the analyzed correlation 13.

In more detail, the phenotype feature analyzing unit 120 determines phenotype features 14 having a distribution of expression levels that indicate a positive correlation or negative correlation between the prognostic indices 11, as the phenotype features 14 in which the correlation 13 exists, using the obtained statistical data.

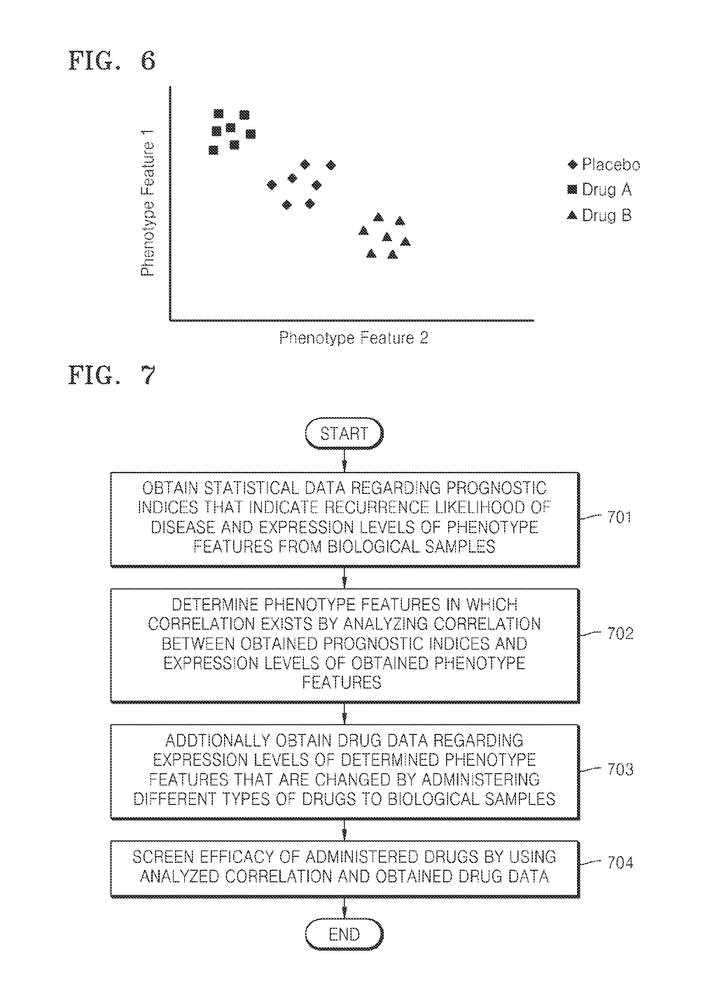

FIG. 5 is a graph for explaining an operation of analyzing the correlation 13 between the prognostic indices 11 and the phenotype features 12 using the phenotype feature analyzing unit 120, according to an embodiment of the present disclosure. Referring to FIG. 5, the distribution of expression levels of the phenotype features 12, such as phenotype feature 1, phenotype feature 2, and phenotype feature 3 in relation to the distribution of the prognostic indices 11 (recurrence scores) is shown.

In phenotype feature 1, as the recurrence score increases, the expression level of phenotype feature 1 increases. Thus, the phenotype feature analyzing unit 120 analyzes that the recurrence score and phenotype feature 1 are in proportion to each other and a positive correlation between the recurrence score and phenotype feature 1 exists.

In phenotype feature 2, as the recurrence score increases, the expression level of phenotype feature 2 decreases. Thus, the phenotype feature analyzing unit 120 analyzes that the recurrence score and phenotype feature 2 are in inverse proportion to each other and a negative correlation between the recurrence score and phenotype feature 2 exists.

In phenotype feature 3, since the expression level of phenotype feature 3 is changed regardless of a change of the recurrence score, the phenotype feature analyzing unit 120 analyzes that no correlation 13 between the recurrence score and phenotype feature 3 exists.

As a result, the phenotype feature analyzing unit 120 determines the phenotype features 14 having the positive or negative correlation, such as phenotype features 1 and 2, among various phenotype features 12 of the biological samples 20.

Referring back to FIG. 2, next, various drugs for drug screening, such as drug A, drug B, and drug C, are administered to the biological samples 20. As described above, each drug may cause a reaction for suppressing or increasing gene expression levels of the biological samples 20 or may not cause any reaction. In the present embodiment, unlike the biological samples 20 before drug administration is performed, after drug administration is performed, the biological samples will be referred to with reference numeral 30 (i.e., biological samples 30).

The expression level analyzing unit 112 additionally obtains drug data regarding the expression levels of the phenotype features 14 that are changed and determined by administering different types of drugs to the biological samples 30.

The imaging device, such as a high content cell imaging device, a high content screening device, or a high throughput screening device, is used to observe the expression levels of the phenotype features 14 that are changed by administering the drugs on the biological samples 30.

Here, the phenotype features 14 to be observed correspond to features that are determined by the phenotype feature analyzing unit 120 that the correlation 13 exists in the phenotype features 14. That is, since the determined phenotype features 14 are analyzed as relating to the prognostic indices 11 (a recurrence score), the phenotype features 14 are considered to relate to a malignant tumor.

Although, according to the related art, image data is obtained using a high content cell imaging device, a high content screening device, or a high throughput screening device, it is difficult to precisely analyze the meaning of a change of features or the efficacy of a drug by drug administration. In other words, it is difficult to precisely analyze features that clinically relate to a disease, among many features included in the image data.

However, in the drug screening system 1 of FIG. 1, since the phenotype features 14 relating to the high recurrence likelihood of the disease are sorted from the biological samples 20 and are analyzed, the phenotype features 14 regarding the malignant tumor may be concentratively analyzed. Thus, experimental results may not be greatly affected by the ratio of the malignant tumor with respect to abnormal tissue samples extracted from a patient.

FIG. 6 is a graph showing drug data regarding expression levels of the phenotype features 14 that are changed and determined by administering different types of drugs to the biological samples 30, according to an embodiment of the present disclosure. The drug data of FIG. 6 is the result that is analyzed based on the image data obtained by the expression level analyzing unit 112.

Referring to FIG. 6, a change trend of expression levels of the phenotype features 14, such as phenotype features 1 and 2, which have been determined by administration of the drugs A and B, is shown. The change trend is information that indicates whether the expression levels of the phenotype features 14, such as phenotype features 1 and 2, which have been determined by administration of the drugs A and B, increase or decrease based on a placebo.

Referring back to FIG. 2, the drug screening unit 130 screens the efficacy of the drugs A and B administered by using the correlation 13 that is analyzed by the phenotype feature analyzing unit 120 and the drug data that is obtained by the expression level analyzing unit 112.

In more detail, the drug screening unit 130 screens the administered drugs A and B by predicting a change of the prognostic indices 11 (a recurrence score) that correspond to the change trend of the expression levels of the phenotype features 14, such as phenotype features 1 and 2, which are determined by the drugs A and B, using the analyzed correlation 13.

The drug screening unit 130 screens a drug that is administered to the biological samples 20 wherein the predicted change is that the prognostic indices 11 (a recurrence score) will decrease, as a drug having efficacy.

That is, when the expression levels of the phenotype features 14 determined to have a positive correlation decrease or the expression levels of the phenotype features 14 determined to have a negative correlation increase, the drug screening unit 130 may predict that the prognostic indices 11 (recurrence score) will decrease. For understanding, the following description will be provided with reference to FIG. 6.

Referring to the drug data of FIG. 6, as a result of administering drug A to sample A, it is analyzed that phenotype feature 1 increases compared to the placebo and phenotype feature 2 decreases compared to the placebo. According to the correlation 13 analyzed by the phenotype feature analyzing unit 120, phenotype feature 1 is analyzed to have a positive correlation between phenotype feature 1 and the recurrence score, and phenotype feature 2 is analyzed to have a negative correlation between phenotype feature 2 and the recurrence score.

As a result, the drug screening unit 130 may analyze that drug A causes an increase in the recurrence score compared to the placebo, using the analyzed correlation 13.

Referring back to the drug data of FIG. 6, as a result of administering drug B to sample B, it is analyzed that phenotype feature 1 decreases compared to the placebo and phenotype feature 2 increases compared to the placebo. According to the correlation 13 analyzed by the phenotype feature analyzing unit 120, phenotype feature 1 is analyzed to have a positive correlation between phenotype feature 1 and the recurrence score, and phenotype feature 2 is analyzed to have a negative correlation between phenotype feature 2 and the recurrence score.

As a result, the drug screening unit 130 may analyze that drug B causes a decrease in the recurrence score compared to the placebo, by using the analyzed correlation 13.

It is well known that, as the recurrence score increases, the recurrence likelihood of the disease (e.g., breast cancer) increases. Thus, the drug screening unit 130 may predict that administration of drug A will cause an increase in the recurrence score, based on the above results. Thus, the drug screening unit 130 may screen drug A as a drug having no efficacy as an anticancer drug. However, the drug screening unit 130 may predict that administration of drug B will cause a decrease in the recurrence score, based on the above results. Thus, the drug screening unit 130 may screen drug B as a drug having efficacy as an anticancer drug.

The drug screening unit 130 screens the efficacy of the administered drugs, such as drug A, drug B, and drug C, using the above-described method so as to generate a drug profile 15 regarding the efficacy of the administered drugs, such as drug A, drug B, and drug C.

Therefore, according to the drug screening system 1 of FIG. 1, since the efficacy of various types of drugs may be precisely screened using image data obtained from samples regarding abnormal tissues (e.g., a cancer, a tumor, and the like) extracted from a patient, the drug screening system 1 of FIG. 1 may be used in developing new drugs, preventing a disease, or providing an optimum treatment method to a patient at an early stage of a disease.

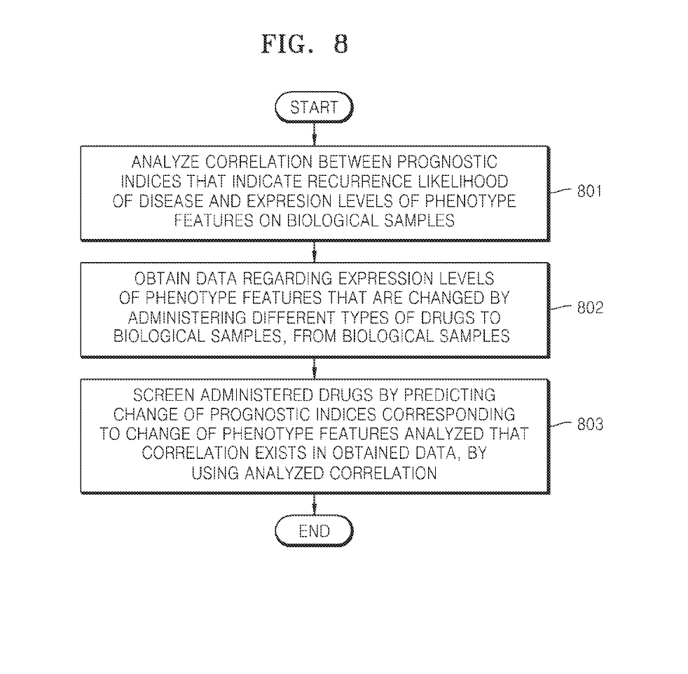

FIG. 7 is a flowchart illustrating a method of performing drug screening, according to an embodiment of the present disclosure. Referring to FIG. 7, the method of performing drug screening, according to the current embodiment of the present disclosure, includes operations to be performed in a time sequence using the drug screening system 1 of FIG. 1 and the apparatus 10 for performing drug screening of FIG. 2. Thus, although omitted below, the above descriptions regarding FIGS. 1 and 2 also apply to the method of performing drug screening illustrated in FIG. 7.

In operation 701, the data obtaining unit 110 obtains statistical data regarding the prognostic indices 11 (a recurrence score, a tumor grade, and the like) that indicate the recurrence likelihood of a disease and expression levels of the phenotype features 12 from the biological samples 20.

In operation 702, the phenotype feature analyzing unit 120 determines phenotype features 14 in which a correlation 13 exists, by analyzing the correlation 13 between the obtained prognostic indices 11 and the expression levels of the phenotype features 12.

In operation 703, the data obtaining unit 110 additionally obtains drug data regarding the expression levels of the phenotype features 14 that are changed and determined by administering different types of drugs to the biological samples 30.

In operation 704, the drug screening unit 130 screens the efficacy of the administered drugs using the analyzed correlation 13 and the obtained drug data.

FIG. 8 is a flowchart illustrating a method of performing drug screening, according to another embodiment of the present disclosure. Referring to FIG. 8, the method of performing drug screening, according to the current embodiment of the present disclosure, includes operations to be performed in a time sequence using the drug screening system 1 of FIG. 1 and the apparatus 10 for performing drug screening of FIG. 2. Thus, although omitted below, the above descriptions regarding FIGS. 1 and 2 also apply to the method of performing drug screening illustrated in FIG. 8.

In operation 801, the phenotype feature analyzing unit 120 analyzes the correlation 13 between the prognostic indices 11 (a recurrence score, a tumor grade, and the like) that indicate the recurrence likelihood of a disease and the expression levels of the phenotype features 12 from the biological samples 20.

In operation 802, the data obtaining unit 110 obtains data regarding the expression levels of the phenotype features 14 that are changed by administering different types of drugs to the biological samples 30.

In operation 803, the drug screening unit 130 predicts a change of the prognostic indices 11 that correspond to a change of the phenotype features 14 analyzed that the correlation 13 exists in the phenotype features 14, from the obtained data, using the analyzed correlation 13, and screens the administered drugs.

The embodiments of the present disclosure can be written as computer programs and can be implemented in general-use digital computers that execute the programs using a computer-readable recording medium. The structure of data used in the embodiments of the present disclosure can be recorded on the computer-readable recording medium by using several units. Examples of the computer-readable recording medium include magnetic storage media (e.g., ROM, floppy disks, hard disks, etc.), optical recording media (e.g., CD-ROMs, or DVDs), and storage media (e.g., transmission through the Internet).

As described above, according to the one or more of the above embodiments of the present disclosure, since the efficacy of various types of drugs can be precisely screened using image data obtained from samples regarding an abnormal tissue (a cancer, a tumor, and the like) extracted from a patient, the present disclosure can be used in developing new drugs, preventing a disease, or providing an optimum treatment method to a patient at an early stage of a disease.

It should be understood that the exemplary embodiments described therein should be considered in a descriptive sense only and not for purposes of limitation. Descriptions of features or aspects within each embodiment should typically be considered as available for other similar features or aspects in other embodiments.

What is claimed:

1. A computer implemented method of performing drug screening, the method comprising the steps, implemented in a processor, of:
obtaining statistical data regarding prognostic indices that indicate a recurrence likelihood of a disease and expression levels of phenotype features from biological samples;
determining phenotype features in which a correlation exists by analyzing the correlation between the obtained prognostic indices and the obtained expression levels;
obtaining drug data regarding expression levels of the determined phenotype features that are changed by administering different types of drugs to the biological samples; and
screening efficacy of the administered drugs using the analyzed correlation and the obtained drug data.

2. The method of claim 1, wherein the determining phenotype features comprises determining phenotype features having a distribution of expression levels that indicate a positive correlation or a negative correlation, in relation to a distribution of the prognostic indices, as the phenotype features in which the correlation exists, using the obtained statistical data.

3. The method of claim 1, wherein the screening efficacy of the administered drugs comprises screening the administered drugs by predicting a change of the prognostic indices that correspond to a change trend of the expression levels of the determined phenotype features by the administered drugs, using the analyzed correlation.

4. The method of claim 3, wherein the screening efficacy of the administered drugs comprises screening a drug administered to the biological samples wherein the predicted change is that the prognostic indices will decrease, as a drug having efficacy.

5. The method of claim 4, wherein the screening efficacy of the administered drugs comprises predicting that the prognostic indices will decrease when expression levels of phenotype features determined to have a positive correlation decrease or expression levels of phenotype features determined to have a negative correlation increase.

6. The method of claim 1, wherein the obtained drug data comprises a change trend of the expression levels of the phenotype features determined by the administered drugs, and the change trend comprises information that indicates whether the expression levels of the phenotype features determined by the administered drugs increase or decrease based on a placebo.

7. The method of claim 1, wherein the obtaining drug data and the screening of the administered drugs are performed using image data regarding the biological samples to which the drugs are administered.

8. The method of claim 7, wherein the image data is obtained using at least one of a high content cell imaging technique, a high content screening technique, and a high throughput screening technique.

9. The method of claim 1, wherein the biological samples comprise samples that comprise an abnormal tissue and that are transfected by small interfering RNA (siRNA) so that the biological samples have different gene expression levels.

10. The method of claim 1, wherein the biological samples comprise samples that correspond to different cell lines comprising an abnormal tissue.

11. The method of claim 1, further comprising estimating the prognostic indices using gene expression data obtained from the biological samples, wherein the obtained statistical data comprises data obtained from the estimated prognostic indices, and the obtained gene expression data is obtained using at least one of reverse transcriptase polymerase chain reaction (RT-PCR) and a microarray.

12. The method of claim 1, wherein the prognostic indices use at least one of a recurrence score, a tumor grade, and a C-path score.

13. The method of claim 1, further comprising analyzing the phenotype features of the biological samples using image data in which phenotypes expressed in the biological samples are indicated, wherein the obtained statistical data comprises data obtained from the analyzed phenotype features.

14. A computer implemented method of performing drug screening, the method comprising the steps, implemented in a processor, of:
analyzing a correlation between prognostic indices that indicate a recurrence likelihood of a disease and expression levels of phenotype features on biological samples;
obtaining data regarding expression levels of the phenotype features that are changed by administering different types of drugs to the biological samples; and
screening the administered drugs by predicting a change of the prognostic indices that correspond to a change of the phenotype features analyzed that the correlation exits in the obtained data, using the analyzed correlation.

15. The method of claim 14, wherein the screening of the administered drugs comprises screening a drug administered to the biological samples wherein the predicted change is that the prognostic indices will decrease, as a drug having efficacy.

16. A non-transitory computer-readable recording medium having recorded thereon a program for executing a method of performing drug screening, which when executed by a processor causes the processor to:
obtain statistical data regarding prognostic indices that indicate a recurrence likelihood of a disease and expression levels of phenotype features from biological samples;
determine phenotype features in which a correlation exists by analyzing the correlation between the obtained prognostic indices and the obtained expression levels;
obtain drug data regarding expression levels of the determined phenotype features that are changed by administering different types of drugs to the biological samples; and
screen efficacy of the administered drugs using the analyzed correlation and the obtained drug data.

17. An apparatus for performing drug screening, the apparatus comprising:
a data obtaining unit that obtains statistical data regarding prognostic indices that indicate a recurrence likelihood of a disease and expression levels of phenotype features from biological samples;
a phenotype feature analyzing unit that determines phenotype features in which a correlation exists by analyzing the correlation between the obtained prognostic indices and the obtained expression levels; and
a drug screening unit that screens efficacy of administered drugs using the analyzed correlation and drug data regarding expression levels of the determined phenotype features that are changed by administering different types of drugs to the biological samples, wherein the drug data is obtained by the data obtaining unit.

18. The apparatus of claim 17, wherein the phenotype feature analyzing unit determines phenotype features having a distribution of expression levels that indicate a positive correlation or a negative correlation, in relation to a distribution of the prognostic indices, as the phenotype features in which the correlation exists, using the obtained statistical data.

19. The apparatus of claim 17, wherein the drug screening unit screens the administered drugs by predicting a change of the prognostic indices that corresponds to a change trend of the expression levels of the determined phenotype features by the administered drugs, by using the analyzed correlation.

20. The apparatus of claim 19, wherein the drug screening unit screens a drug administered to the biological samples wherein the predicted change is that the prognostic indices will decrease, as a drug having efficacy.

21. The apparatus of claim 20, wherein the drug screening unit predicts that the prognostic indices will decrease when expression levels of phenotype features determined to have a positive correlation decrease or expression levels of phenotype features determined to have a negative correlation increase.

22. The apparatus of claim 17, wherein the data obtaining unit and the drug screening unit use image data obtained from the biological samples to which the drugs are administered, wherein the image data is obtained using at least one of a high content cell imaging technique, a high content screening technique, and a high throughput screening technique.

23. The apparatus of claim 17, wherein the biological samples comprise at least one of samples that comprise an abnormal tissue and that are transfected by small interfering RNA (siRNA) so that the biological samples have different gene expression levels and samples that correspond to different cell lines comprising an abnormal tissue.

24. The apparatus of claim 17, wherein the data obtaining unit comprises a prognostic index estimating unit that estimates the prognostic indices using gene expression data obtained from the biological samples, and the obtained statistical data comprises data obtained from the estimated prognostic indices, and the obtained gene expression data are obtained using at least one of reverse transcriptase polymerase chain reaction (RT-PCR) and a microarray.

25. The apparatus of claim 17, wherein the prognostic indices use at least one of a recurrence score, a tumor grade, and a C-path score.

26. The apparatus of claim 17, wherein the data obtaining unit comprises an expression level analyzing unit that analyzes the phenotype features of the biological samples using image data in which phenotypes expressed in the biological samples are indicated, and the obtained statistical data comprises data obtained from the analyzed phenotype features.

27. An apparatus for performing drug screening, the apparatus comprising:
a phenotype feature analyzing unit that analyzes a correlation between prognostic indices that indicate a recurrence likelihood of a disease and expression levels of phenotype features on biological samples;
a data obtaining unit that obtains data regarding expression levels of the phenotype features that are changed by administering different types of drugs to the biological samples; and
a drug screening unit that screens the administered drugs by predicting a change of the prognostic indices that correspond to a change of the phenotype features analyzed that the correlation exits in the obtained data, using the analyzed correlation.

28. The apparatus of claim 27, wherein the drug screening unit screens a drug administered to the biological samples wherein the predicted change is that the prognostic indices will decrease, as a drug having efficacy.
